(19) United States
(12) Patent Application Publication
(10) Pub. No.: US 2014/0316530 A1
(43) Pub. Date: Oct. 23, 2014

(54) Metatarsalphalangeal Joint Apprartus and Method

(71) Applicant: John S. Early, Dallas, TX (US)

(72) Inventor: John S. Early, Dallas, TX (US)

(73) Assignee: Bespa, Inc

(21) Appl. No.: 13/868,582

(22) Filed: Apr. 23, 2013

(51) Int. Cl. A61F 2/42

(52) U.S. Cl. CPC A61F 2/4225
USPC 623/21.19

(57) ABSTRACT

A metatarsalphalangeal joint apparatus and method includes a metatarsal stem conformed to make cortical contact at the isthmus of a metatarsal bone and metatarsal head conformed to connect with the metatarsal stem. A phalangeal stem is conformed to make cortical contact at the isthmus of a phalangeal bone and a phalangeal base is conformed to connect with the phalangeal stem. A bearing surface is provided between the metatarsal head and the phalangeal base.

### FIELD OF THE INVENTION

This invention relates to a metatarsalphalangeal joint apparatus and method. In particular, in accordance with one embodiment, the invention relates to a metatarsalphalangeal joint apparatus consisting of a metatarsal stem conformed to make cortical contact at the isthmus of a metatarsal bone and a metatarsal head conformed to connect with the metatarsal stem. A phalangeal stem is conformed to make cortical contact at the isthmus of a phalangeal bone and a phalangeal base is conformed to connect with the phalangeal stem. A bearing surface is provided between the metatarsal head and the phalangeal base.

### BACKGROUND OF THE INVENTION

A problem exists with regard to repair and/or reconstruction of damaged metatarsalphalangeal joints. In particular, but not by way of limitation, the first metatarsalphalangeal joints (the “big toe” joint”) are important for normal walking gait patterns in humans. When the first metatarsalphalangeal joint of the foot becomes arthritic, for example only, the normal joint motion needed for normal walking is lost. This creates significant pain and loss of function for the individual. One prior art solution which is widely accepted for this condition is a fusion of the joint where the position of the joint is fixed so that the patient can walk with reduced pain. This ‘solution’, however, does not restore any motion to the joint.

There have been a number of prior art attempts to design and manufacture an artificial surface to put in place of the natural joint to restore both motion and function. These attempts have included resurfacing techniques such as metal caps, single surface coverings of either the metatarsal head or the base of the proximal phalanx, or multi component implants to create a total replacement joint. To date, however, there is not a device yet acceptable as a durable and reliable device for use in the human forefoot. The present state of the art tries to faithfully recreate the original anatomy of the joint but prior art devices lack sufficient ability to maintain stability in the high stress environment of the foot. The main problem is that soft tissue changes occurring as the initial disease was progressing, for example again, lead to permanent changes that do not allow restoration of the original anatomy.

Thus, there is a need in the art for a metatarsalphalangeal joint device that is designed for use with damaged joints and which provides essentially normal, pain free range of motion. It therefore is an object of this invention to provide a metatarsalphalangeal joint apparatus and method for repair and restoration of a metatarsalphalangeal joint that results in essentially normal and pain free motion. It is a further object to provide a metatarsalphalangeal joint device that restores stability to the joint, that is adaptable to accommodate differences in size and location and is easy to install and easy to maintain after installation.

### SUMMARY OF THE INVENTION

Accordingly, the metatarsalphalangeal joint apparatus of the present invention, according to one embodiment, includes a metatarsal stem conformed to make cortical contact at the isthmus of a metatarsal bone and a metatarsal head conformed to connect with the metatarsal stem. A phalangeal stem is conformed to make cortical contact at the isthmus of a phalangeal bone and a phalangeal base is conformed to connect with the phalangeal stem. A bearing surface is provided between the metatarsal head and the phalangeal base.

All terms used herein are given there common meaning as known in the art. Thus, for example, “bearing surface” identifies a surface upon which another object rests or bears. As used herein, it identifies material with a smooth surface made of hard material resistant to wear such as polyethylene plastic, preferably medical grade, for example only. Also, it is inherent that metatarsal bones include a base, a shaft and a head. The shaft typically has an ‘isthmus’ or narrow section. Further, bones inherently include a hard ‘cortical’ exterior with a softer interior. Thus, according to the present invention, the metatarsal stem and the phalangeal stem are designed to be inserted within these bones and, from the inside, to make cortical contact at the isthmus of both these bones. This important element of the invention is unique to the art in which this feature is absent and which is actually advocated against. In combination with the other elements of the invention, however, Applicant has found it highly effective in providing stability to the device, among other things.

In another aspect, the metatarsal head is conformed to receive the bottom of a metatarsal bone and a portion of the top of a metatarsal bone. As used herein, the term “a portion” indicates that not all of the top of the metatarsal bone is present within the metatarsal head. As will be discussed more fully hereafter, some of the top of the metatarsal head is removed to reduce contact with the phalangeal component.

In one aspect, the phalangeal base includes an attachment device conformed to receive the bearing surface and to hold the bearing surface such the bearing surface extends beyond the cortical rim of the phalangeal bone.

In another aspect, the metatarsal head is formed in an open arc and the phalangeal base is formed in a bulbous protrusion shape conformed to fit within the open arc. As used herein, the term “open arc” describes a concave surface with an opening such as presented by a deep spoon bowl, for example. In one aspect, the bearing surface is conformed to connect with the metatarsal head within the open arc. In a further aspect, this metatarsal head is also conformed to receive the bottom of a metatarsal bone and a portion of the top of a metatarsal bone.

In one aspect, the metatarsal head includes a first end and a second end with an opening from the second end toward the first end where the opening is larger at the second end and tapers to smaller at the first end; and where the phalangeal stem includes a first end and second end and an opening from the first end toward the second end and where the opening is larger at the first end and tapers to smaller at the second end; and further where the metatarsal stem and the phalangeal head include a tapered projection where the metatarsal stem tapered projection is conformed to fit within the metatarsal head opening and where the phalangeal head tapered projection is conformed to fit within the phalangeal stem opening.

In other aspects, the metatarsal stem includes a transverse hole and/or the phalangeal stem includes a transverse hole. The term “transverse hole” describes a hole that passes through the stem or stems and, as will be described more fully hereafter, is used to further secure the stems in place if needed with a screw, for example only.

According to another embodiment of the invention, a metatarsalphalangeal joint apparatus includes a metatarsal stem with a first end and a second end, the metatarsal stem conformed to make cortical contact at the isthmus of a metatarsal bone where the metatarsal stem includes a tapered projection. A metatarsal head is provided with an opening at a first end and a second end with an opening extending toward the first end and where the opening is larger at the second end. The tapered projection of the metatarsal stem is conformed to connect with the metatarsal head within the opening and the metatarsal head is conformed to receive the bottom of a metatarsal bone and a portion of the top of a metatarsal bone. A phalangeal stem with a first end and a second end is conformed to make cortical contact at the isthmus of a phalangeal bone where the phalangeal stem includes an opening at the first end and extending toward the second end and where the opening is larger at the first end. A phalangeal base with a tapered projection is provided where the tapered projection is conformed to connect with the phalangeal stem within the opening and a bearing surface is provided between the metatarsal head and the phalangeal base.

In one aspect, the phalangeal base is conformed to fit within the cortical rim of a phalangeal bone and includes an attachment device conformed to receive the bearing surface and to hold the bearing surface such the bearing surface extends beyond the cortical rim. In another aspect, the attachment device consists of extended arms within which a portion of the bearing surface is captured.

In a further aspect, the metatarsal head is formed in an open arc and the phalangeal base is formed in a bulbous protrusion shape conformed to fit within the open arc. In one aspect, the bearing surface is conformed to connect with the metatarsal head within the open arc.

In another aspect, the metatarsal stem includes a transverse hole and the phalangeal stem includes a transverse hole.

In one aspect, the bearing surface is medical grade polyethylene.

According to another embodiment, a metatarsalphalangeal joint replacement method includes:

a. providing a metatarsal stem and inserting it in a metatarsal bone such that the metatarsal stem makes cortical contact at the isthmus of the metatarsal bone;

b. connecting a metatarsal head with the metatarsal stem;

c. providing a phalangeal stem and inserting it in a phalangeal bone to make cortical contact at the isthmus of the phalangeal bone;

d. connecting a phalangeal base with the phalangeal stem;

e. connecting a bearing surface between the metatarsal head and the phalangeal base and

f. positioning the metatarsal head next to the phalangeal base with the bearing surface in between.

In one aspect, the metatarsal head is conformed to receive the bottom of a metatarsal bone and a portion of the top of a metatarsal bone.

In another aspect, the phalangeal base includes an attachment device conformed to receive the bearing surface and hold the bearing surface such the bearing surface extends beyond the cortical rim of the phalangeal bone.

In a further aspect, the metatarsal head is formed in an open arc and the phalangeal base is formed in a bulbous protrusion shape conformed to fit within the open arc and the bearing surface is conformed to connect with the metatarsal head within the open arc.

### DESCRIPTION OF THE DRAWINGS

Other objects, features and advantages of the present invention will become more fully apparent from the following detailed description of the preferred embodiment, the appended claims and the accompanying drawings in which:

FIG. 1 is a top partial cut away view of the metatarsal phalangeal joint of the present invention according to one embodiment;

FIG. 2 is a side partial cut away view of the invention of FIG. 1;

FIG. 3 is a close up of the bottom of the metatarsal head of FIG. 1 with grooves for the sesamous;

FIG. 4 is a side view of just the metatarsal head and the phalangeal head of the invention of FIG. 1;

FIG. 5 is a top view of the bearing surface of the invention;

FIG. 6 is a top view of the phalangeal head of the invention of FIG. 1 showing extended arms holding the bearing surface in connection with the phalangeal head;

FIG. 7 is back view of the metatarsal head of the invention of FIG. 1 taken along line A-A in FIG. 4;

FIG. 8 is top partial cut away view of the metatarsalphalangeal joint of the present invention according to another embodiment;

FIG. 9 is a side partial cut away view of the invention of FIG. 8;

FIG. 10 is a side view of just the metatarsal head and the phalangeal head of the invention of FIG. 8;

FIG. 11 is a top view of metatarsal head connected with the phalangeal head of the invention of FIG. 8;

FIG. 12 is a back view of the metatarsal head of the invention of FIG. 8; and

FIG. 13 is a front view of the metatarsal head of the invention of FIG. 8 taken along line A-A of FIG. 10.

### DETAILED DESCRIPTION OF THE INVENTION

The preferred embodiment of the present invention is illustrated by way of example in FIGS. 1-13. With specific reference to FIGS. 1 and 2, the metatarsalphalangeal joint apparatus 10, according to one embodiment, includes a metatarsal stem 12 with a first end 14 and a second end 16. The metatarsal stem 12 is selected to match whatever joint is selected so that the metatarsal stem 12 makes contact with the cortical 18 at the isthmus 20 of metatarsal bone 22 from the inside 24 of metatarsal bone 22 as shown.

In one embodiment, metatarsal stem 12 includes a tapered projection 26, shown in dotted lines in FIGS. 1 and 2, at first end 14. In one aspect, hole 28 is provided in metatarsal stem 12. Hole 28 enables a screw or pin, not shown, to pass through metatarsal stem 12 and secure it in place if deemed necessary.

Metatarsal head 30 is provided and includes a first end 32 and a second end 34. Opening 36 is formed in second end 34 and tapers in diameter toward first end 32. Importantly, metatarsal stem 12 is conformed to include the bottom 38 of metatarsal bone 22 and just a portion of the top 40 of metatarsal bone 22. Preferably, a section 42 (shown in dotted lines in FIG. 2) of the top 40 of metatarsal bone 22 is removed such that, again, the bottom 38 and the remaining portion of the top 40 of metatarsal bone 22 is contained within metatarsal head 30.

Phalangeal stem 44 has a first end 46 and a second end 48 and extends far enough into phalangeal bone 50 so as to make contact with the cortical 18 at the isthmus 20 from the inside 52 of phalangeal bone 50. According to one embodiment, phalangeal stem 44 includes an opening 54 in the first end 46 and extending toward second end 48. Opening 54 preferably is larger at first end 46 and tapers toward second end 48.

Hole 56 may be provided through phalangeal stem 44 so as to allow a screw or pin 58 (see FIG. 1) to be used to secure phalangeal stem 44 in place and, thus, serves the same purpose as hole 28 in metatarsal stem 12.

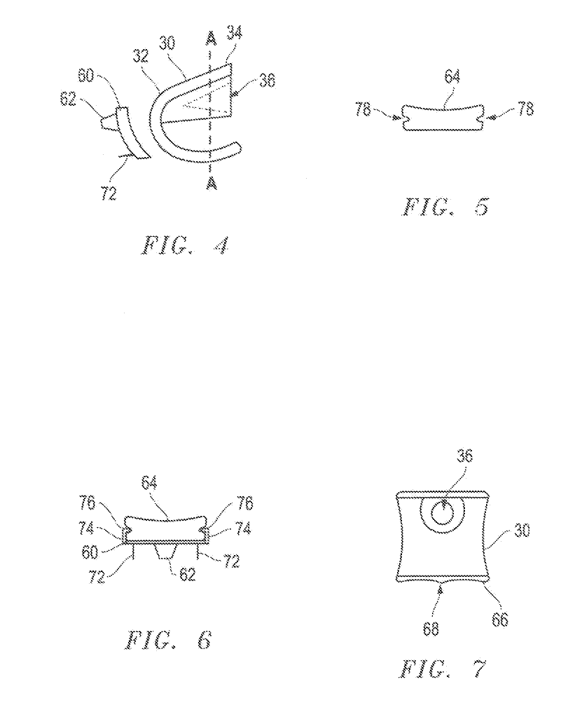

Still referring to FIGS. 1 and 2, phalangeal base 60 is connected with phalangeal stem 44. Preferably, phalangeal base 60 includes a tapered projection 62 (more clearly shown in FIG. 4). Tapered projection 62 is conformed to fit within opening 54 in phalangeal stem 44 and be secured therein.

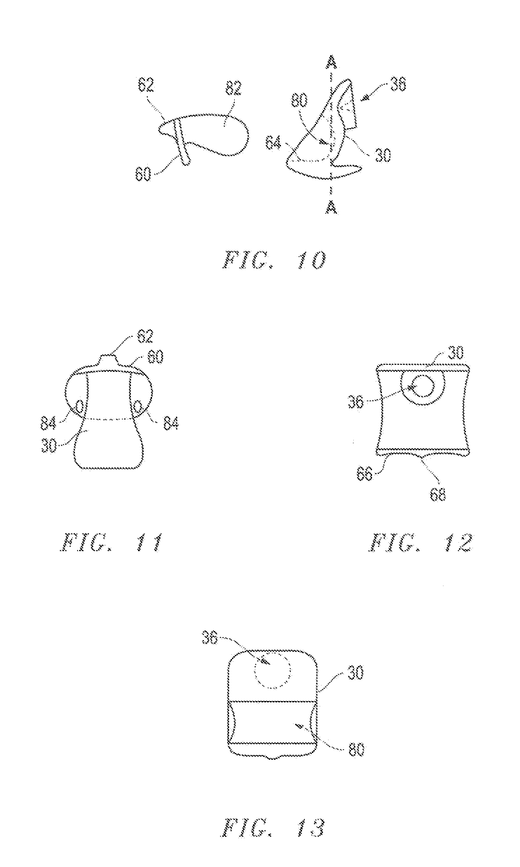

Bearing surface 64 is located between metatarsal head 30 and phalangeal base 60. Preferably, bearing surface 64 is connected with either phalangeal base 60 as shown in FIGS. 1 and 2 or with metatarsal head 30 as shown in FIG. 10.

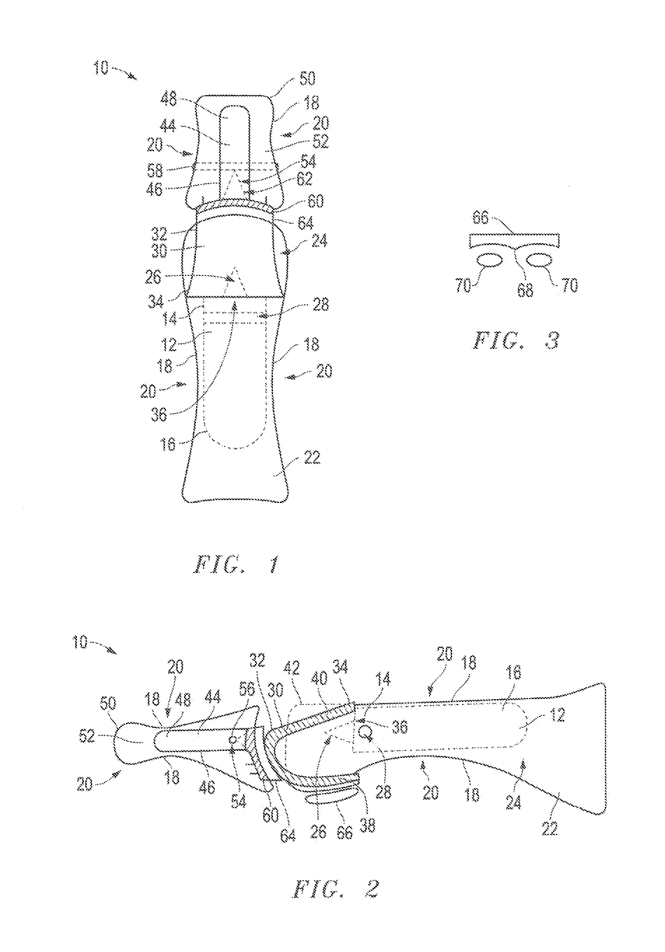

Referring now to FIG. 3, the bottom 66 of metatarsal head 30 is shown to include cristae 68 within which sesamous 70 may lay so as to help restore stability to the plantar plate (not shown).

FIG. 4 isolates important elements of the invention for clarity. Metatarsal head 30 with first end 32 and second end 34 shows tapered opening 36. Also shown is phalangeal base 60 with tapered projection 62. Phalangeal base 60 may also include pegs 72 if desired. Pegs 72 stabilize phalangeal base with the native bone of phalangeal bone 50.

FIG. 5 illustrates the separate nature of bearing surface 64. That is, it is designed to be connected and replaced when worn with ease without need to remove other elements of the invention. FIG. 6 shows an embodiment where phalangeal base 60 includes arms 74 extending from phalangeal base 60. Arms 74 may include rails 76 that fit within slots 78 (best seen in FIG. 5) in bearing surface 64. As can be understood, it is a simple matter to slide bearing surface 64 slots 78 along rails 76 of arms 74 so as to removably connect bearing surface 64 with phalangeal base 60.

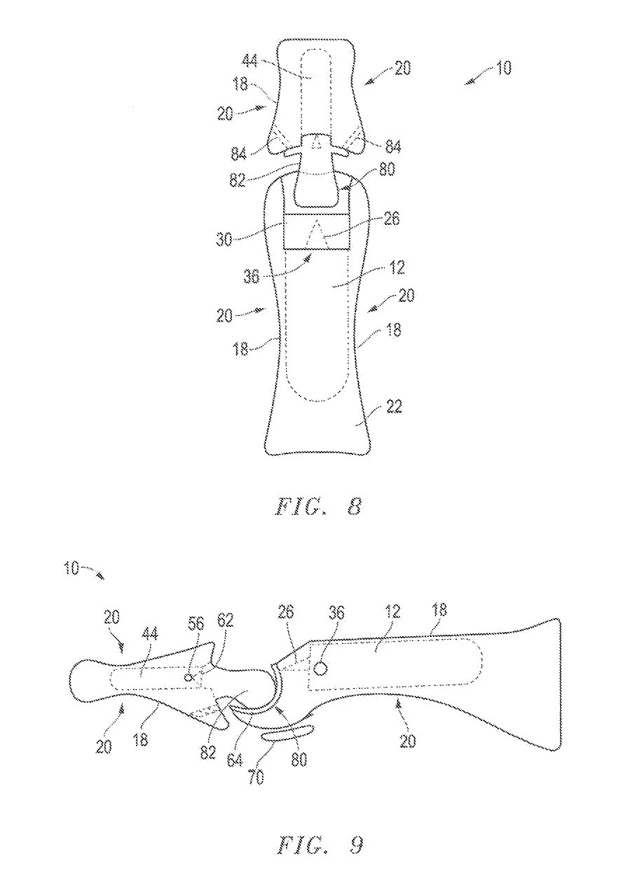

FIG. 7 is a sectional view taken along line A-A of FIG. 4 and shows the back of metatarsal head 30. Opening 36 is clearly shown along with the cristae 68 in the bottom 66.

Referring now to FIGS. 8 and 9 another embodiment of metatarsalphalangeal joint apparatus 10 is shown. The same numbers used with the discussion of the apparatus above are used in all the figures where appropriate. Thus, metatarsal stem 12 includes a first end 14 and second end 16 and is conformed to make contact with the cortical 18 at the isthmus 20 of metatarsal bone 22 from the inside 24 of metatarsal bone 22 as shown. Likewise, phalangeal stem 44 includes a first end 46 and a second end 48 and is conformed to make contact with the cortical 18 at the isthmus 20 of phalangeal bone 50 from the inside 52 of phalangeal bone 50 as shown. Further, holes 28 and 56 are provided if needed as before. In this embodiment, however, metatarsal head 30 and phalangeal base 60 are different in structure. Here, metatarsal head 30 is formed in an open arc 80. The open arc 80 faces up from the bottom 66 of metatarsal head 30 and provides a contact surface that has a concave, cylindrical arc which allows it to capture the phalangeal base 60 into a fixed axial position. In this embodiment, phalangeal base 60 includes bulbous protrusion 82 that fits within and is captured by open arc 80. Screws 84 are provided to secure phalangeal base 60 in place along with tapered projection 62 in opening 54 of phalangeal stem 44 as discussed above. Similarly, metatarsal head 30 includes opening 36 that cooperates with tapered projection 26 of metatarsal stem 12 to secure metatarsal head 30 in place as before.

In this embodiment, bearing surface 64 is conformed to connect with open arc 80. It may connect so as to be removably connectable in any useful way such as with snaps or glue or press fitting or the like. Phalangeal base 60 with bulbous protrusion 82 is clearly shown along with tapered projection 62

Referring now to FIG. 10, metatarsal head 30 formed with open arc 80 is shown along with opening 30 in second end 34. FIGS. 11, 12 and 13 show the top, back and front views of the connected elements of FIG. 10.

By way of continued description, preferably the metatarsal stem 12 is a solid sintered metal component which has a tapered end 16 designed to have a tight cortical 18 fit with the isthmus 20 of the metatarsal bone 22 and a morse taper female end to fit securely with the head component 30. It will come in a number sizes varied by length and end diameter to meet the anatomical variances of the metatarsal bone 22. There will be an optional hole 28 through the diameter just distal to the taper which can be used to pass a screw through for added stabilization.

The metatarsal head 30 will have two iterations that will offer two different bearing surfaces depending on surgeon preference. Both components will be designed to resurface the sesamoid articular surface with a centerline cristae 68 of appropriate size to support the sesamoid position and capture the plantar cortical bone of the metatarsal. Each will provide a phalangeal bearing surface that has a smaller total arc than the native bone to reduce tissue strain. The first iteration will have a shape resembling the bottom 2/3 of the normal metatarsal head architecture. The top part will be at an angle to reduce contact with the phalangeal component and change the motion from a sliding one to a hinge or motion about a contact point. The second iteration is a reverse arc head. It continues to have the sesamoid and cortical lock components described earlier but the joint contact surface has a concave, cylindrical arc allowing it to capture the phalangeal component into a fixed axial position. This second component will have a snap fit polyethylene bearing surface 64 within the concave metal surface. The heads will also be designed to engage the underside of the dorsal cortex of the first metatarsal at the point of true cortical bone to provide stability to the articular surface. They will be provided in a variety of sizes to accommodate different anatomy including right and left, and in special circumstances for bone loss. Both metatarsal head components 30 will leave native bone on the medial and lateral sides distally to allow soft tissue reattachment and aid in the stability of the joint.

The phalangeal stem 44 will preferably be a solid, sintered metal component with a tapered end to engage the cortex 18 at the neck 20 of the proximal phalanx 50 and a opening 54 for morse taper at the other end for a secure fit with the phalangeal base component. There will be an optional hole 56 through the diameter of the stem 44 just beyond the morse taper for added stabilization. It will be provided in sizes to accommodate the varied anatomy.

The phalangeal base component 60 will preferably be made in two styles to mate with the two different metatarsal head 30 configurations. The base 60 for style one will have a sintered metal base with the male end of a morse taper to fit into the stem 44. It will be designed to fit within the cortical rim of the bone 50 leaving only the polyethylene bearing surface 64 exposed. The plantar base will have pegs or screws 72 to stabilize the plate with the native bone. There will be a medial, lateral and plantar “u” arms 74 to facilitate capture of the polyethylene bearing surface 64. There will be a dorsal lip, rails 76, to “lock” the bearing surface 64 in place.

The phalangeal base 60 for the second head design will have the male end, tapered section 62, for the morse taper of the stem 44 also. The base 60 will be designed to have cortical 18 contact with the phalanx 50 when implanted. The plantar base 60 will have two screw holes for screws 84 securing the base 60 to the native bone. The articulating metal head, bulbous protrusion 82, will be shaped to fit within the concave confines of open arc 80 the matched metatarsal head 30. Preferably there will be a right and left side to this implant as the cylindrical head 82 will have an offset to allow some translational motion along the articular surface of open arc 80. The offset is useful to block motion away from the lesser toes.

Preferably the bearing surface 64 is made of medical grade polyethylene and the polyethylene components will be two different styles to compliment the articulations of the matched metatarsal head 30 and phalangeal base 60 components. Importantly, the polyethylene insert 64 is designed to be replaceable without removal of the implants. The implants will be designed to accommodate different polyethylene thicknesses for the option shown in FIGS. 1-7. There will be groves, slots 78, in the sides and bottom of the bearing surface 64 to lock in the tracts, rails 76, provided by the phalangeal base 60. Option two, the option shown in FIGS. 8-13, will be a fixed bearing surface 64 thickness but also provide a snap fit, or the like, so as to be replaceable.

By way of continued explanation, the method of implantation of the metatarsalphalangeal joint 10 is through a dorsal approach to the first metatarsophalangeal joint. The extensor hallicus longus sheath is protected as the incision is carried deep medially to the tendon. The joint is exposed and dissection is done sub periosteally to expose the whole metatarsal head. The medial and lateral collateral ligaments are released. With the proximal phalanx now plantar flexed ninety degrees, the plantar plate is released from the metatarsal head to gain complete access for implantation. The base of the proximal phalanx is removed with a saw to expose the cancellous core. Phalangeal broaches are used to determine the appropriate phalangeal stem 44 for implant. The trial is left in place for preparing the base.

The metatarsal shaft is then located with a guide wire by fluoroscopy. Cannulated broaches are used to size up the canal for the correct implant. The final broach is left in place. Returning to the proximal phalanx the appropriate cutting/shaping jig is placed onto the phalanx base and secured to the trial stem left in place. The shaping jig is then removed leaving the trial stem in place.

The appropriate shaping jig for the metatarsal head 30 is then placed onto the trail broach on the metatarsal 22 and secured. The head of the metatarsal 22 is shaped to fit the metatarsal head 30 configuration chosen for the patient. The jig is then removed and the trial joint is inserted to assess range of motion and stability. Adjustments are made to position and cuts until the surgeon is satisfied.

If the first metatarsal head 30 configuration is used, the appropriate implants for the phalanx 50 are implanted first. The metatarsal shaft 12 and head 30 follow. If the second iteration is used the metatarsal head 30 should be implanted first followed by the phalanx.

The description of the present embodiments of the invention has been presented for purposes of illustration, but is not intended to be exhaustive or to limit the invention to the form disclosed. Many modifications and variations will be apparent to those of ordinary skill in the art. As such, while the present invention has been disclosed in connection with an embodiment thereof, it should be understood that other embodiments may fall within the spirit and scope of the invention as defined by the following claims.

What is claimed is:

1. A metatarsalphalangeal joint apparatus comprising:
a. a metatarsal stem conformed to make cortical contact at the isthmus of a metatarsal bone;
b. a metatarsal head conformed to connect with said metatarsal stem;
c. a phalangeal stem conformed to make cortical contact at the isthmus of a phalangeal bone;
d. a phalangeal base conformed to connect with said phalangeal stem; and
e. a bearing surface between said metatarsal head and said phalangeal base.

2. The apparatus of claim 1 wherein said metatarsal head is conformed to receive the bottom of a metatarsal bone and a portion of the top of a metatarsal bone.

3. The apparatus of claim 1 wherein said phalangeal base includes an attachment device conformed to receive said bearing surface and hold said bearing surface such said bearing surface extends beyond the cortical rim of said phalangeal bone.

4. The apparatus of claim 1 wherein said metatarsal head is formed in an open arc and said phalangeal base is formed in a bulbous protrusion conformed to fit within said open arc.

5. The apparatus of claim 4 wherein said bearing surface is conformed to connect with said metatarsal head within said open arc.

6. The apparatus of claim 4 wherein said metatarsal head is conformed to receive the bottom of a metatarsal bone and a portion of the top of a metatarsal bone.

7. The apparatus of claim 1 wherein said metatarsal head includes a first end and a second end with an opening from said second end toward said first end wherein said opening is larger at said second end and tapers to smaller at said first end; and wherein said phalangeal stem includes a first end and second end and an opening from said first end toward said second end and wherein said opening is larger at said first end and tapers to smaller at said second end; and further wherein said metatarsal stem and said phalangeal head include a tapered projection wherein said metatarsal stem tapered projection is conformed to fit within said metatarsal head opening and wherein said phalangeal head tapered projection is conformed to fit within said phalangeal stem opening.

8. The apparatus of claim 1 wherein said metatarsal stem includes a transverse hole.

9. The apparatus of claim 1 wherein said phalangeal stem includes a transverse hole.

10. A metatarsalphalangeal joint apparatus comprising:
a. a metatarsal stem with a first end and a second end, said metatarsal stem conformed to make cortical contact at the isthmus of a metatarsal bone wherein said metatarsal stem includes a tapered projection at said proximal end;
b. a metatarsal head with a first end and a second end with an opening at said second end and extending toward said first end and wherein said opening is larger at said first end wherein said tapered projection is conformed to connect with said metatarsal stem within said opening and wherein said metatarsal head is conformed to receive the bottom of a metatarsal bone and a portion of the top of a metatarsal bone;
c. a phalangeal stem with a first end and a second end, said phalangeal stem conformed to make cortical contact at the isthmus of a phalangeal bone wherein said phalangeal stem includes an opening at said first end and extending toward said second end and wherein said opening is larger at said first end;
d. a phalangeal base with a tapered projection wherein said tapered projection is conformed to connect with said phalangeal stem within said opening; and
e. a bearing surface between said metatarsal head and said phalangeal base.

11. The apparatus of claim 10 wherein said phalangeal base is conformed to fit within the cortical rim of a phalangeal bone and includes an attachment device conformed to receive said bearing surface and hold said bearing surface such said bearing surface extends beyond said cortical rim.

12. The apparatus of claim 11 wherein said attachment device consists of extended arms within which a portion of said bearing surface is captured.

13. The apparatus of claim 10 wherein said metatarsal head is formed in an open arc and said phalangeal base is formed in a bulbous protrusion conformed to fit within said open arc.

14. The apparatus of claim 13 wherein said bearing surface is conformed to connect with said metatarsal head within said open arc.

15. The apparatus of claim 10 wherein said metatarsal stem includes a transverse hole and wherein said phalangeal stem includes a transverse hole.

16. The apparatus of claim 10 wherein said bearing surface is medical grade polyethylene.

17. A metatarsalphalangeal joint replacement method comprising:
a. providing a metatarsal stem and inserting it in a metatarsal bone such that said metatarsal stem makes cortical contact at the isthmus of said metatarsal bone;
b. connecting a metatarsal head with said metatarsal stem;
c. providing a phalangeal stem and inserting it in a phalangeal bone to make cortical contact at the isthmus of said phalangeal bone;
d. connecting a phalangeal base with said phalangeal stem;
e. connecting a bearing surface between said metatarsal head and said phalangeal base. and
f. positioning said metatarsal head next to said phalangeal base with said bearing surface in between.

18. The method of claim 17 wherein said metatarsal head is conformed to receive the bottom of a metatarsal bone and a portion of the top of a metatarsal bone.

19. The method of claim 17 wherein said phalangeal base includes an attachment device conformed to receive said bearing surface and hold said bearing surface such said bearing surface extends beyond the cortical rim of said phalangeal bone.

20. The method of claim 17 wherein said metatarsal head is formed in an open arc and said phalangeal base is formed in a bulbous protrusion conformed to fit within said open arc and wherein said bearing surface is conformed to connect with said metatarsal head within said open arc.
